(19) United States
(12) Patent Application Publication
(10) Pub. No.: US 2018/0326198 A1
(43) Pub. Date: Nov. 15, 2018

(54) FEMALE CONNECTOR

(71) Applicant: JMS CO., LTD., Hiroshima-shi, Hiroshima (JP)

(72) Inventors: Takehiko YUKI, Hiroshima (JP); Takahiko KUNISHIGE, Hiroshima (JP); Yasumasa UEHARA, Hiroshima (JP); Kazuhiko TAKIMOTO, Hiroshima (JP)

(21) Appl. No.: 15/776,535

(22) PCT Filed: Dec. 14, 2016
(86) PCT No.: PCT/JP2016/087167

§ 371 Date: May 16, 2018

(30) Foreign Application Priority Data
Dec. 15, 2015 (JP) 2015-244431
Aug. 25, 2016 (JP) 2016-164765

(51) Int. Cl. A61M 39/10; A61J 15/00

(52) U.S. Cl. CPC A61M 39/1011; A61M 39/16; A61J 15/0053

(57) ABSTRACT

A female connector (1) is connectable to a male connector (100) including a cylindrical male member (921). The female connector (1) includes: a tubular portion (11) having a hollow circular cylindrical shape to which the male member (921) is insertable; and a cylindrical portion (17) having a hollow circular cylindrical shape that is coaxial with the tubular portion (11) and surrounded by the tubular portion (11). When the male member (921) is inserted into the tubular portion (11), a liquid-tight seal (19) is formed between the cylindrical portion (17) and the male member (921), and the cylindrical portion (17) and the male member (921) communicate with each other.

### TECHNICAL FIELD

The present invention relates to a female connector preferably used for enteral feeding.

### BACKGROUND ART

Enteral feeding is known as a method for administering a liquid including a nutrient, a drug and the like to a patient who cannot take food orally. In enteral feeding, a catheter is indwelled in the patient in a state of being inserted into an alimentary canal (e.g., stomach) from the outside of the body. Examples of the catheter include a transnasal catheter that is inserted through the patient's nose and a PEG (Percutaneous Endoscopic Gastrostomy) catheter that is inserted into a gastrostomy formed in the patient's abdomen. Liquids such as nutrients, liquid food (generally called “enteral nutrients”), and drugs are administered to the patient via such catheters. In administration of the liquid to the patient, a container that stores the liquid and a catheter (e.g., a transnasal catheter, a PEG catheter) that is indwelled in the patient are connected via a flexible tube, etc. Conventionally, in order to connect different members, a male connector is used as a connector on the upstream side (container side) of the liquid flow (hereinafter, referred to as “container-side connector”), and a female connector is used as a connector on the downstream side (patient side) of the liquid flow (hereinafter, referred to as “patient-side connector”) (for example, see Patent Document 1).

In order to avoid misconnection with connectors used in fields other than enteral feeding, recently international standard ISO 80369-3 regarding nutrition-related medical equipment has been given consideration for the international standardization of a female connector 910 shown in FIGS. 12A and 12B as the container-side connector and a male connector 920 shown in FIGS. 13A and 13B as the patient-side connector.

The female connector (container-side connector) 910 shown in FIGS. 12A and 12B includes a tubular portion (female member) 911 having a hollow circular cylindrical shape. An inner circumferential face 912 of the tubular portion 911 is a tapered face whose inner diameter increases toward the tip (a so-called female tapered face). A spiral protrusion (male threading) 915 is formed on the outer circumferential face of the tubular portion 911.

The male connector (patient-side connector) 920 shown in FIGS. 13A and 13B has a cylindrical male member 921 and an outer tube 923 that surrounds the male member 921. The male member 921 and the outer tube 923 are coupled via a base plate 924 that projects in a flange shape along the radial direction from the base end of the male member 921. An outer circumferential face 922 of the male member 921 is a tapered face whose outer diameter decreases toward the tip (a so-called male tapered face). In the male member 921, a channel 927 is formed that passes through the male member 921 along the lengthwise direction. An inner circumferential face 928 of the male member 921 that defines the channel 927 is a circular cylindrical face whose inner diameter is constant along the lengthwise direction of the male member 921. Female threading 925 is formed on the inner circumferential face of the outer tube 923 that faces the male member 921.

The female connector 910 and the male connector 920 are connected by inserting the male member 921 into the tubular portion 911 and screwing the spiral protrusion 915 with the female threading 925 as shown in FIG. 14. Since the inner circumferential face 912 of the tubular portion 911 and the outer circumferential face 922 of the male member 921 are tapered faces having a corresponding diameter and taper angle, they form a liquid-tight seal and come into surface contact with each other. The spiral protrusion 915 and the female threading 925 screwed together constitute screw lock mechanisms for locking the connected state of the female connector 910 and the male connector 920. The female connector 910 and the male connector 920 provide a connection having excellent liquid tightness (property of preventing the leakage of a liquid from the connection portion of the female connector 910 and the male connector 920 even if pressure is applied to the liquid) and excellent connection strength (property of preventing separation of the connected female connector 910 and the male connector 920 even if pulling force is applied).

### PRIOR ART DOCUMENT

### Patent Document

[Patent Document 1] WO 2008/152871

### DISCLOSURE OF INVENTION

### Problem to be Solved by the Invention

When the female connector 910 and the male connector 920 are in the connected state as shown in FIG. 14, a liquid (e.g., an enteral nutrient) flows from the female connector 910 to the male connector 920 (downward in FIG. 14). A space extending from a small diameter portion 913 of the female connector 910 to the channel 927 of the male member 921 (a dotted area in FIG. 14) is filled with the liquid.

Thereafter, the female connector 910 and the male connector 920 are separated. At this time, part of the liquid in the space is attached to a base-end inner circumferential face 912a (see FIG. 12B) of the tubular portion 911 of the female connector 910 and a tip outer circumferential face 922a of the male member 921 of the male connector 920 (see FIGS. 13A and 13B).

The liquid that has attached to the female connector 910 and the male connector 920 after separation is not administered to a patient. Because of this, a connector tool made up of the female connector 910 and the male connector 920 has a problem that an accurate amount of the liquid as measured cannot be administered to a patient. For example, when the liquid contains a drug, the accuracy of the administration amount of the drug to a patient decreases, and the expensive drug will be wasted.

A connector tool not including screw lock mechanisms (the spiral protrusion 915 and the female threading 925) and establishing connection by simply inserting the male member 921 into the tubular portion 911 (so-called slip connection) also has the above problem.

It is an object of the present invention to reduce the amount of the liquid to be attached to a male connector and a female connector after separation of the male connector and the female connector.

### Means for Solving Problem

A female connector of the present invention is connectable to a male connector including a cylindrical male member. The female connector includes: a tubular portion having a hollow circular cylindrical shape to which the male member is insertable; and a cylindrical portion having a hollow circular cylindrical shape that is coaxial with the tubular portion and surrounded by the tubular portion. The cylindrical portion is configured so that, when the male member is inserted into the tubular portion, a liquid-tight seal is formed between the cylindrical portion and the male member, and the cylindrical portion and the male member communicate with each other.

### Effects of the Invention

According to the female connector of the present invention, when the female connector is connected to the male connector, a liquid-tight seal is formed between the cylindrical portion and the male member, and the cylindrical portion and the male member communicate with each other. The seal prevents the liquid from flowing to the inner circumference face of the tubular portion or the outer circumference face of the male member. Thus, it is possible to reduce the amount of the liquid to be attached to the male connector and the female connector after separation of the male connector and the female connector.

### BRIEF DESCRIPTION OF DRAWINGS

FIG. 1A is a perspective view of a female connector according to Embodiment 1 of the present invention, and FIG. 1B is a cross-sectional view of the female connector taken along a plane that includes the central axis of the female connector.

FIG. 2A is a perspective view of a male connector that is connectable to the female connector of the present invention, and FIG. 2B is a cross-sectional view of the male connector taken along a plane that includes the central axis of the male connector.

FIG. 3A is a perspective view showing a state in which the female connector according to Embodiment 1 of the present invention is connected to a male connector.

FIG. 3B is a cross-sectional view of FIG. 3A.

FIG. 4 is a cross-sectional perspective view of a female connector according to Embodiment 2 of the present invention.

FIG. 5 is a cross-sectional view showing a state in which the female connector according to Embodiment 2 of the present invention is connected to a male connector.

FIG. 6 is a cross-sectional view showing a state in which a female connector according to Embodiment 3 of the present invention is connected to a male connector.

FIG. 7 is a cross-sectional view of a female connector according to Embodiment 4 of the present invention.

FIG. 8 is a cross-sectional view showing a state in which the female connector according to Embodiment 4 of the present invention is connected to a male connector.

FIG. 9A is a perspective view of a female connector according to Embodiment 5 of the present invention, and FIG. 9B is a cross-sectional view of the female connector taken along a plane that includes the central axis of the female connector.

FIG. 10 is a cross-sectional view showing a state in which the female connector according to Embodiment 5 of the present invention is connected to a male connector.

FIG. 11 is a cross-sectional view taken along a plane that includes the central axis of a female connector according to Embodiment 6 of the present invention.

FIG. 12A is a perspective view of a female connector under consideration as ISO 80369-3. FIG. 12B is a cross-sectional view of the female connector taken along a plane that includes the central axis of the female connector.

FIG. 13A is a perspective view of a male connector under consideration as ISO 80369-3. FIG. 13B is a cross-sectional view of the male connector taken along a plane that includes the central axis of the male connector.

FIG. 14 is a cross-sectional view showing a state in which a male connector and a female connector under consideration as ISO 80369-3 are connected.

### DESCRIPTION OF THE INVENTION

In one aspect of the female connector of the present invention, the liquid-tight seal can be formed between an outer circumference face of the cylindrical portion and an inner circumference face of the male member. This preferred aspect is advantageous to form a liquid-tight seal with a simple configuration.

An outer circumference face of the cylindrical portion may include a male tapered face whose outer diameter decreases toward a tip. In this case, the liquid-tight seal may be formed between the male tapered face and an inner circumference face of the male member. This preferred aspect is advantageous to easily form a liquid-tight seal with a simple configuration.

The cylindrical portion may be made from a soft material. This preferred aspect is advantageous to relax the accuracy of the cylindrical portion and to improve the liquid tightness of the seal.

In another aspect of the female connector of the present invention, an outer circumference face of the cylindrical portion may have an annular protrusion that is continuous in a circumferential direction of the cylindrical portion. In this case, the liquid-tight seal can be formed between the annular protrusion and an inner circumference face of the male member. This preferred aspect also is advantageous to form a liquid-tight seal with a simple configuration.

In the above, the annular protrusion may be an O-ring that is mounted on the outer circumference face of the cylindrical portion. This preferred aspect is advantageous to produce the annular protrusion that forms a liquid-tight seal with a general-purpose member easily.

When the liquid-tight seal is formed, an inner circumference face of the tubular portion may not be fitted to an outer circumference face of the male member liquid-tightly. This configuration is advantageous to improve the liquid tightness of the seal between the cylindrical portion and the male member and to relax the accuracy of the inner circumferential face of the tubular portion.

When the liquid-tight seal is formed, an inner circumference face of the tubular portion and an outer circumference face of the male member may be spaced apart from each other. This configuration is advantageous to improve the liquid tightness of the seal between the cylindrical portion and the male member and to relax the accuracy of the inner circumferential face of the tubular portion.

The tubular portion may include a slit so as to be elastically deformable to enlarge its diameter. This configuration is advantageous to improve the liquid tightness of the seal between the cylindrical portion and the male member and to relax the accuracy of the inner circumferential face of the tubular portion.

The tubular portion may be made from a soft material. This configuration is advantageous to improve the liquid tightness of the seal between the cylindrical portion and the male member and to relax the accuracy of the tubular portion.

When the liquid-tight seal is formed, an inner circumference face of the tubular portion may be fitted to an outer circumference face of the male member liquid-tightly. This configuration is advantageous to prevent the liquid from passing through a slight gap between the female connector and the male connector to leak outside when the liquid pressure is increased. Further, in a connector tool that does not include screw lock mechanisms for maintaining the connected state of the female connector and the male connector, this configuration is advantageous to maintain the connected state of the female connector and the male connector stably.

The male connector may further include an outer tube that surrounds the male member, and female threading that is provided on the outer tube so as to face the male member. In this case, the female connector may further include a spiral protrusion that is provided on an outer circumference face of the tubular portion so as to be screwed with the female threading. This preferred aspect is advantageous to apply the present invention to a connector tool that includes screw lock mechanisms represented by ISO 80369-3. This preferred aspect is also advantageous to avoid a situation that the male connector becomes unsanitary by the liquid that has flowed into a gap between the male member and the outer tube after separation of the female connector and the male connector.

Hereinafter, the present invention will be described in detail by way of preferred embodiments. Needless to say, the present invention is not limited to the following embodiments. For convenience of explanation, the respective drawings referred to hereinafter are simplified drawings showing exclusively principal members constituting the embodiments of the present invention. The present invention therefore possibly includes arbitrary members not shown in the drawings referred to hereinafter. Further, the respective members shown in the drawings referred to hereinafter may be modified or omitted within the scope of the present invention.

### Embodiment 1

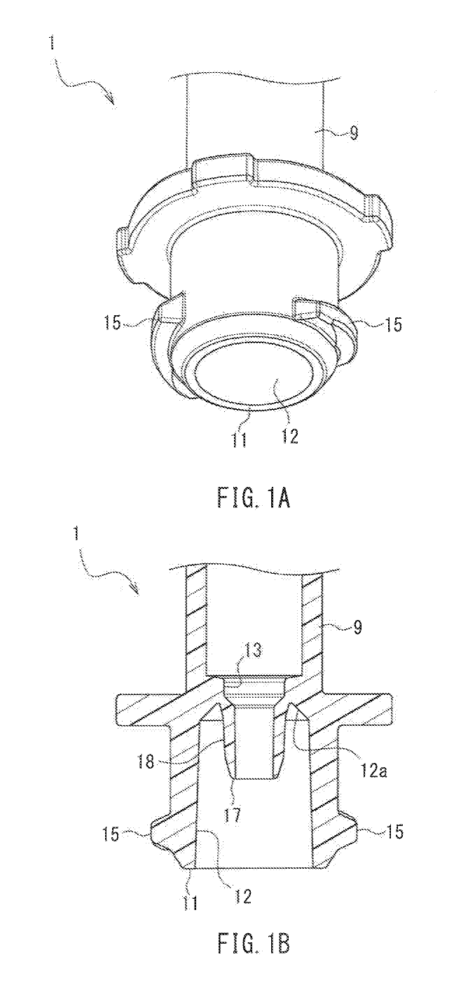

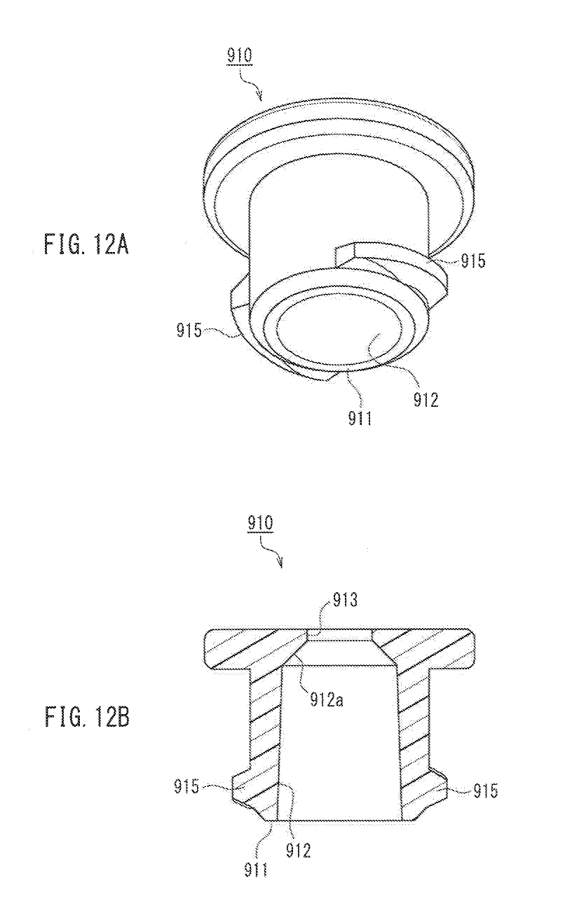

FIG. 1A is a perspective view of a female connector 1 according to Embodiment 1 of the present invention, and FIG. 1B is a cross-sectional view thereof. The female connector 1 includes a tubular portion 11 having a hollow circular cylindrical shape. An inner circumferential face 12 of the tubular portion 11 is a tapered face whose inner diameter increases toward the tip (a so-called female tapered face). A spiral protrusion (male threading) 15 is formed on the outer circumferential face of the tubular portion 11. These members are substantially the same as those of the female connector 910 shown in FIGS. 12A and 12B, and preferably compliant with ISO 80369-3.

As shown in FIG. 1B, the female connector 1 of this embodiment further includes a cylindrical portion 17 having a hollow circular cylindrical shape. The cylindrical portion 17 is provided at a portion of the base end of the tubular portion 11 where the inner diameter is relatively small (a small diameter portion 13). The cylindrical portion 17 is coaxial with the tubular portion 11 and surrounded by the tubular portion 11. The cylindrical portion 17 is spaced apart from the tubular portion 11 in the radial direction. An outer circumferential face 18 of the cylindrical portion 17 includes a tapered face whose outer diameter decreases toward the tip (a so-called male tapered face).

In this embodiment, the female connector 1 is provided at the tip of an outer tube 9 of a syringe (injector) as a casing tip (nozzle). However, the present invention is not limited to this. The female connector of the present invention can be provided in any member. For example, the female connector may be provided at the end of a flexible tube. The configuration on the side opposite to the female connector 1 relative to the small diameter portion 13 (upper side in FIG. 1B) can be modified appropriately.

The female connector 1 is preferably made from a hard material. Examples of the material include, but are not limited to, resin materials such as polycarbonate, polypropylene, polyacetal, polyamide, rigid polyvinyl chloride, polyethylene, styrene ethylene, polyethylene terephthalate, polybutylene terephthalate, and butylene styrene block copolymer. The entire female connector 1 can be formed integrally as a single piece by subjecting any of the above resin materials to injection molding.

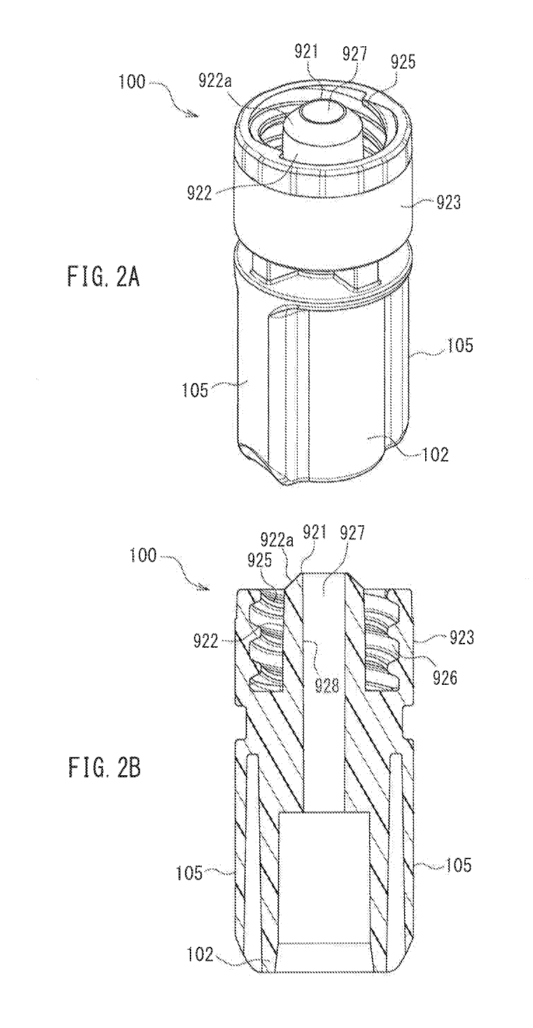

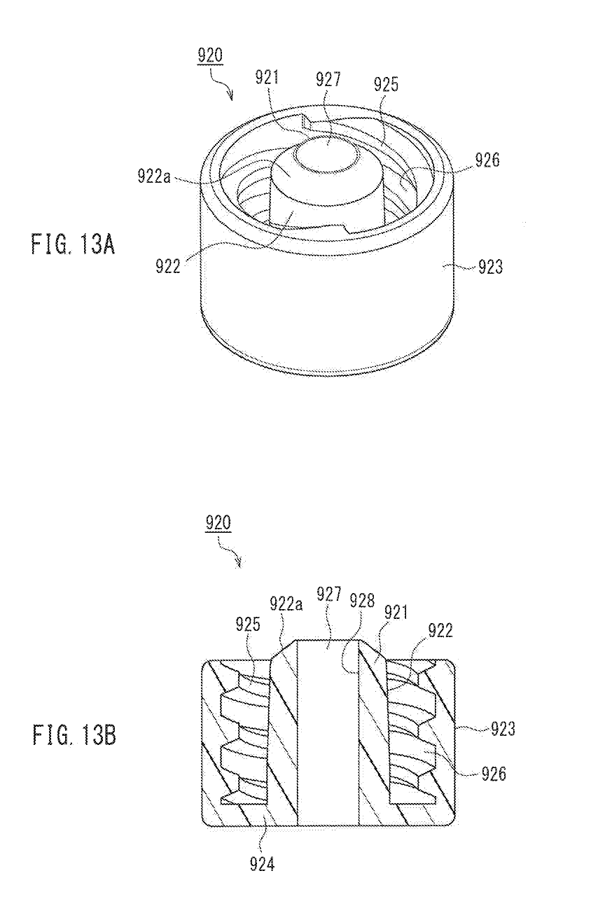

FIG. 2A is a perspective view of a male connector 100, and FIG. 2B is a cross-sectional view thereof. Similarly to the male connector 920 shown in FIGS. 13A and 13B, the male connector 100 is compliant with ISO 80369-3. In FIGS. 2A and 2B, the same reference numerals are assigned to the same components as those of the male connector 920 shown in FIGS. 13A and 13B, and explanations thereof are omitted. The male connector 100 includes a connection portion 102 on the end opposite to the male member 921 side.

As shown in FIG. 2B, the connection portion 102 communicates with a channel 927 that is formed in the male member 921, and the inner circumference face of the connection portion 102 is a circular cylindrical face that is coaxial with the male member 921. Although not illustrated, a flexible tube is inserted and fixed in the connection portion 102. The tube may be a catheter (e.g., a transnasal catheter, a PEG catheter) that is indwelled in a patient, or a tube that is connected to the catheter. A pair of grip portions 105 sandwiches the connection portion 102 so that an operator can hold the male connector 100 easily. Note here that the male connector 100 described herein is only an example, and particularly the shape of the portions of the male connector 100 not specified by ISO 80369-3 can be changed appropriately. For example, the shape of the grip portion 105 may be changed, or the grip portion 105 may be omitted. The connection portion 102 may be inserted into the tube, instead of the tube being inserted into the connection portion 102.

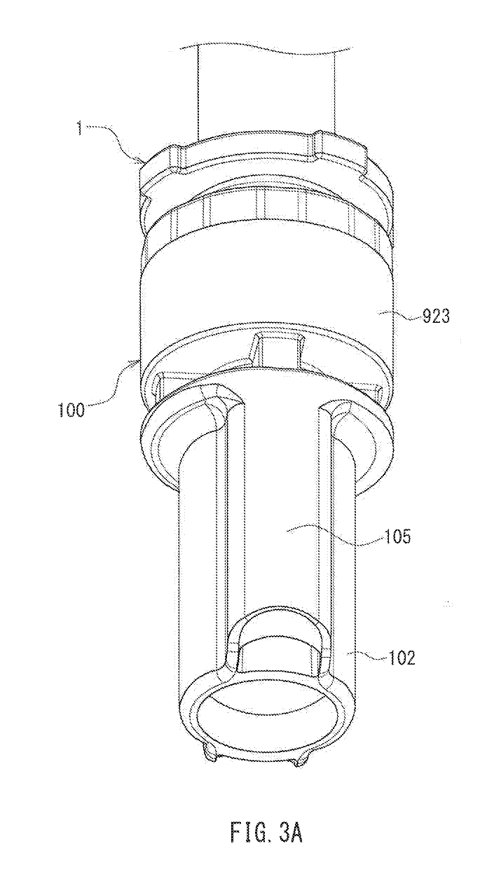

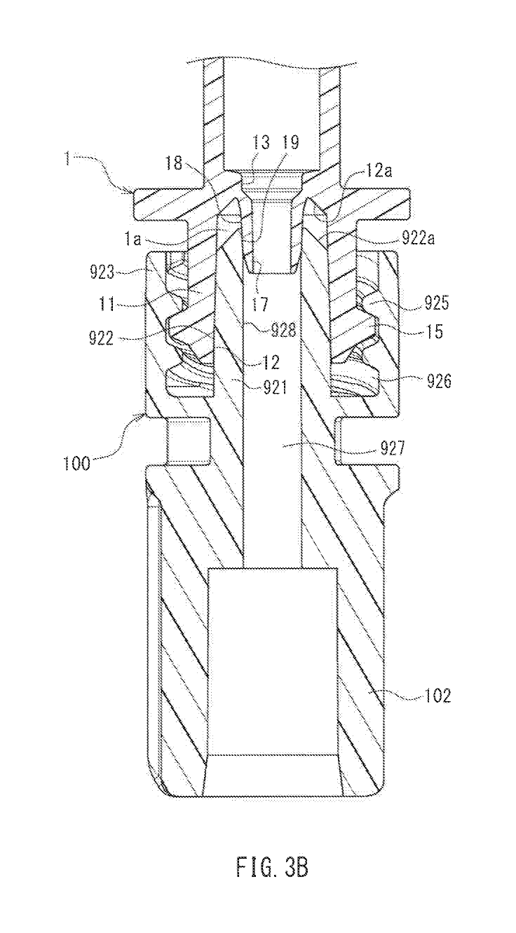

FIG. 3A is a perspective view showing a state in which the female connector 1 is connected to the male connector 100, and FIG. 3B is a cross-sectional view thereof. As shown in FIG. 3B, the cylindrical portion 17 of the female connector 1 is inserted in the male member 921 of the male connector 100. As described above, a male tapered face is provided on the outer circumferential face 18 of the cylindrical portion 17, and the inner circumferential face 928 of the male member 921 is a circular cylindrical face. The minimum outer diameter of the male tapered face of the cylindrical portion 17 on the tip side is smaller than the inner diameter of the inner circumferential face 928 of the male member 921, and the maximum outer diameter of the male tapered face on the base end side is larger than the inner diameter of the inner circumferential face 928. Thus, the male tapered face of the cylindrical portion 17 and the tip portion of the inner circumferential face 928 of the male member 921 (i.e., the opening edge of the channel 927 on the tip side) are fitted to each other, and thereby a liquid-tight seal 19 is formed therebetween. Consequently, the cylindrical portion 17 and the male member 921 communicate with each other liquid-tightly.

The male member 921 of the male connector 100 is inserted in the tubular portion 11 of the female connector 1. The tubular portion 11 is inserted in a gap 926 between the male member 921 and the outer tube 923. The spiral protrusion 15 is screwed with the female threading 925.

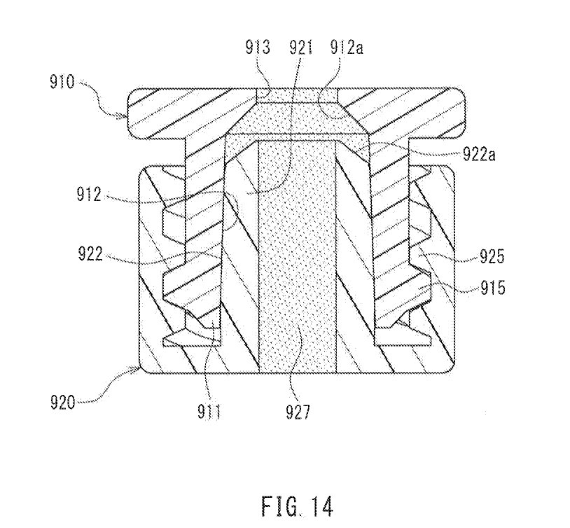

When the female connector 1 and the male connector 100 are in the connected state as shown in FIGS. 3A and 3B, a liquid (e.g., an enteral nutrient) flows from the female connector 1 to the male connector 100 (downward in FIG. 3B). As can be understood by comparing FIG. 3B and FIG. 14, since the liquid-tight seal 19 is formed between the cylindrical portion 17 and the male member 921 in this embodiment, the liquid will not flow into a space 1a surrounded by the tubular portion 11 and the cylindrical portion 17 and the male member 921. Accordingly, when the female connector 1 and the male connector 100 are separated later, the liquid will not be attached to a base-end inner circumferential face 12a of the tubular portion 11 or a tip outer circumferential face 922a of the male member 921. Thus, it is possible to reduce the amount of the liquid to be attached to the female connector 1 and the male connector 100 after separation of the female connector 1 and the male connector 100, as compared with the case of using the female connector 910 shown in FIGS. 12A and 12B.

With this configuration, it becomes possible to administer an accurate amount of the liquid as measured to a patient. When the liquid contains a drug, the administration amount of the drug to a patient can be controlled accurately, and an expensive drug will not be wasted.

As described above, when the conventional female connector 910 (see FIGS. 12A and 12B) is used, a liquid will be attached to the tip outer circumferential face 922a of the male member 921 of the male connector 920 (see FIGS. 13A and 13B) after separation of the female connector 910. This liquid sometimes flows into the gap 926 between the male member 921 and the outer tube 923 (see FIGS. 13A and 13B). Since a spacing between the outer circumferential face 922 of the male member 921 and the female threading 925 of the outer tube 923 is very narrow, it is difficult to wipe away the liquid that has flowed into the gap 926 by inserting a cotton bud, etc., into the gap 926. When the male connector 920 is provided at the upstream end of a PEG catheter that is inserted in a patient, the male connector 920 remains indwelled in the patient over a long period of time (e.g., one to three months) together with the PEG catheter. If the male connector 920 with the liquid remaining inside the gap 926 remains indwelled in the patient over a long period of time, the male connector 920 can become unsanitary. Eventually, bacteria may breed in the male connector 920, enter the patient's body, and cause a serious complication. Meanwhile, in the case of using the female connector 1 of this embodiment, the liquid will not be attached to the tip outer circumferential face 922a of the male connector 100 (see FIGS. 2A and 2B) after separation of the female connector 1. This solves the above problem, which can occur in the case of using the female connector 910 (see FIGS. 12A and 12B), that the liquid flows into the gap 926 between the male member 921 and the outer tube 923, and the male connector becomes unsanitary.

In the above example, although the entire female connector 1 is constituted by the same material (e.g., hard material), the present invention is not limited to this. For example, the cylindrical portion 17 may be constituted by a soft material. Examples of the soft material include, but are not limited to: resin materials such as polypropylene (PP) and polyethylene (PE); and materials having rubber elasticity (also called elastomer) such as rubbers including natural rubber, isoprene rubber and silicone rubber, and thermoplastic elastomers including styrene-based elastomer, olefin-based elastomer, polyurethane-based elastomer and vinyl chloride-based elastomer. The portions of the female connector 1 other than the cylindrical portion 17 (hereinafter, the portions are referred to as a body portion) can be constituted by any of the above hard materials. The method for combining the cylindrical portion 17 and the body portion made from different materials is not limited, and may be a method including uniting the cylindrical portion 17 and the body portion by coinjection molding (coinjection molding method), a method including producing the cylindrical portion 17 and the body portion separately and connecting them liquid-tightly (connection method), or the like. In the connection method, any method can be used to connect the cylindrical portion 17 and the body portion that are produced separately, such as a method using an adhesive, a method applying fusion, or the like. The cylindrical portion 17 made from a soft material is advantageous to relax the accuracy of the cylindrical portion 17 and to improve the liquid tightness of the seal 19.

The male tapered face provided on the outer circumferential face 18 of the cylindrical portion 17 is advantageous in that the liquid-tight seal 19 is formed easily between the outer circumferential face 18 of the cylindrical portion 17 and the inner circumferential face 928 of the male member 921 with a simple configuration. Note here that the outer circumferential face 18 may have a shape other than the male tapered face. For example, the outer circumferential face 18 of the cylindrical portion 17 may be a circular cylindrical face whose outer diameter is constant along the lengthwise direction. In this case, by managing the accuracy of the outer circumferential face 18 properly, it is possible to bring the outer circumferential face 18 of the cylindrical portion 17 and the inner circumferential face 928 of the male member 921 into surface contact with each other and form a liquid-tight seal therebetween.

Constituting the cylindrical portion 17 by any of the above soft materials is advantageous to improve the liquid tightness of the seal.

The inner circumferential face (female tapered face) 12 of the tubular portion 11 may or may not be fitted to the outer circumferential face (male tapered face) 922 of the male member 921 liquid-tightly when the seal 19 is formed between the male tapered face provided on the outer circumferential face 18 of the cylindrical portion 17 and the tip portion of the inner circumferential face 928 of the male member 921 (see FIG. 3B).

In the first configuration example, the seal 19 is formed between the cylindrical portion 17 and the male member 921, and almost at the same time, the inner circumferential face 12 of the tubular portion 11 and the outer circumferential face 922 of the male member 921 are fitted to each other liquid-tightly and the liquid-tight seal is formed therebetween. Since a plurality of seals are formed between the female connector 1 and the male connector 100, the first configuration example is advantageous to prevent the liquid from passing through a slight gap between the female connector 1 and the male connector 100 to leak outside when the liquid pressure is increased.

In the above first configuration example, it is necessary to simultaneously achieve the fitting of the cylindrical portion 17 and the male member 921 and the fitting of the inner circumferential face 12 and the outer circumferential face 922. If one of the fittings is achieved earlier, it becomes difficult to achieve the other fitting later. To cope with this, in the second configuration example, the inner circumferential face 12 of the tubular portion 11 is not fitted to the outer circumferential face 922 of the male member 921 liquid-tightly (e.g., the inner circumferential face 12 is slightly spaced apart from the outer circumferential face 922) when the seal 19 is formed between the cylindrical portion 17 and the male member 921. This second configuration example is advantageous to relax the accuracy of the inner circumferential face 12 and to improve the liquid tightness of the seal 19 (see Embodiments 4 and 5 described later).

### Embodiment 2

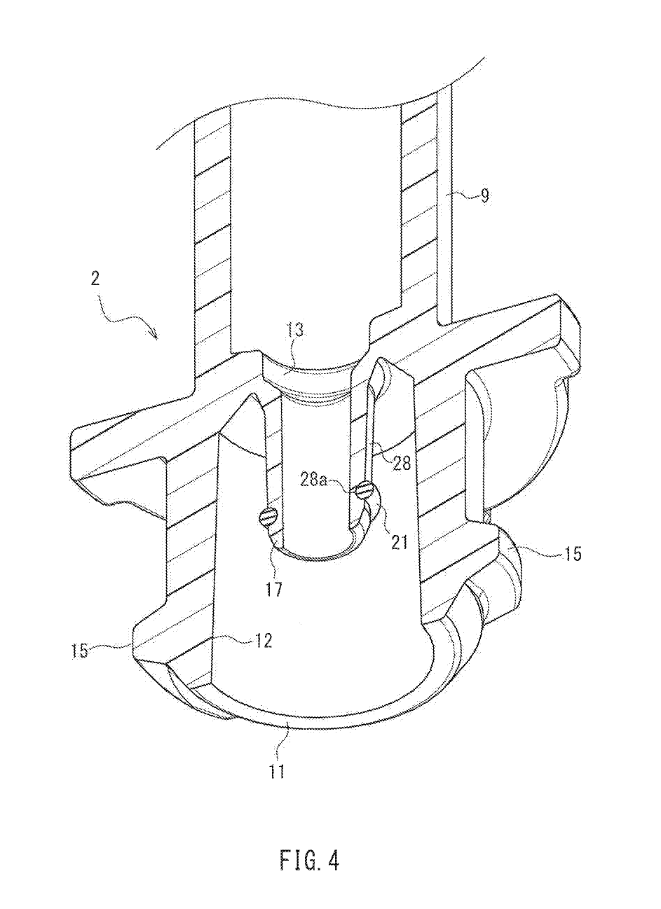

FIG. 4 is a cross-sectional perspective view of a female connector 2 according to Embodiment 2 of the present invention. In the drawings referred to hereinafter, the same reference numerals as those in the drawings of Embodiment 1 are assigned to members corresponding to the members illustrated in the drawings referred to in Embodiment 1. Although the explanations of such members are not repeated in this embodiment, they should be taken into account appropriately. Hereinafter, Embodiment 2 will be described mainly in terms of differences from Embodiment 1.

In the female connector 2 of Embodiment 2, an annular protrusion 21 is provided on an outer circumferential face 28 of the cylindrical portion 17. The annular protrusion 21 is continuous in the circumferential direction of the cylindrical portion 17 and protrudes toward the tubular portion 11. In this embodiment, an O-ring constitutes the annular protrusion 21. An O-ring 21 is fitted in an annular grove 28a that is formed on the outer circumferential face 28 of the cylindrical portion 17 and continuous in the circumferential direction.

The O-ring constituting the annular protrusion 21 may be a general-purpose O-ring that can form a liquid-tight seal. The material of the O-ring is not limited, and preferably a soft material. Examples of the soft material include materials having rubber elasticity (also called elastomer) such as rubbers including natural rubber, isoprene rubber and silicone rubber, and thermoplastic elastomers including styrene-based elastomer, olefin-based elastomer, polyurethane-based elastomer and vinyl chloride-based elastomer.

In this embodiment, the outer circumferential face 28 of the cylindrical portion 17 is a tapered face whose outer diameter decreases toward the tip (a so-called male tapered face), similarly to the outer circumferential face 18 of Embodiment 1. However, in this embodiment, the outer circumferential face 28 does not need to be a male tapered face, and may be, e.g., a circular cylindrical face whose outer diameter is constant along the lengthwise direction of the cylindrical portion 17.

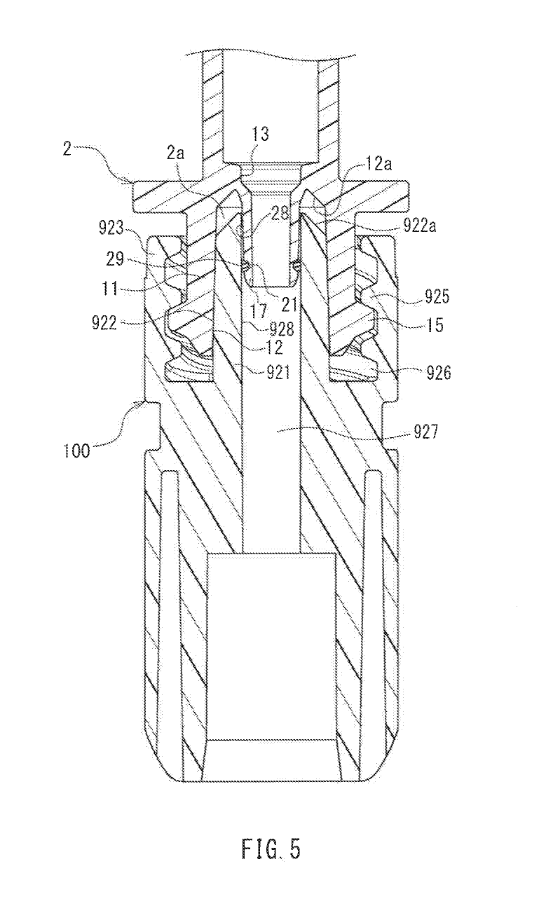

Similarly to Embodiment 1, the female connector 2 can be connected to the male connector 100 (see FIGS. 2A and 2B). FIG. 5 is a cross-sectional view showing a state in which the female connector 2 is connected to the male connector 100. The cylindrical portion 17 of the female connector 2 is inserted in the male member 921 of the male connector 100. The outer circumferential face 28 of the cylindrical portion 17 and the inner circumferential face 928 of the male member 921 are spaced apart from each other in the radial direction owing to a difference in diameter therebetween. The annular protrusion 21 on the outer circumferential face 28 of the cylindrical portion 17 comes into close contact with the inner circumferential face 928 of the male member 921, thereby forming a liquid-tight seal 29 between the annular protrusion 21 and the inner circumferential face 928. In other words, the annular protrusion 21 blocks a gap between the outer circumferential face 28 and the inner circumferential face 928 liquid-tightly. Consequently, the cylindrical portion 17 and the male member 921 communicate with each other liquid-tightly.

Similarly to Embodiment 1, when the female connector 2 and the male connector 100 are in the connected state as shown in FIG. 5, a liquid (e.g., an enteral nutrient) flows from the female connector 2 to the male connector 100 (downward in FIG. 5). Similarly to Embodiment 1, since the liquid-tight seal 29 is formed between the cylindrical portion 17 and the male member 921, the liquid will not flow into a space 2a surrounded by the tubular portion 11 and the cylindrical portion 17 and the male member 921. Accordingly, when the female connector 2 and the male connector 100 are separated later, the liquid will not be attached to the base-end inner circumferential face 12a of the tubular portion 11 or the tip outer circumferential face 922a of the male member 921. Therefore, it is possible to reduce the amount of the liquid to be attached to the female connector 2 and the male connector 100 after separation of the female connector 2 and the male connector 100, as compared with the case of using the female connector 910 shown in FIGS. 12A and 12B. Thus, effects similar to those of Embodiment 1 are achieved.

The annular protrusion 21 can be constituted by a member other than the O-ring. For example, the annular protrusion 21 can be formed integrally with the cylindrical portion 17 on the outer circumferential face of the cylindrical portion 17 by coinjection molding method, etc., using any of the soft materials described in Embodiment 1. This configuration eliminates a step of mounting an O-ring to the cylindrical portion 17.

The cylindrical portion 17 may be constituted by a soft material. Further, the entire cylindrical portion 17 including the annular protrusion 21 may be constituted by a soft material. In these cases, the soft materials to be used can be the same as those described in Embodiment 1. Similarly to the cylindrical portion 17 described in Embodiment 1, the cylindrical portion 17 made from the soft material is combined with the portions of the female connector 2 other than the cylindrical portion 17 (body portion).

Two or more annular protrusions 21 may be provided at different positions in the lengthwise direction of the cylindrical portion 17. In this case, each annular protrusion 21 may be configured to come into close contact with the inner circumferential face 928 of the male member 921 to form the liquid-tight seal 29. This configuration is advantageous to reduce a possibility of the liquid leakage to the space 2a even if the liquid pressure is increased.

The inner circumferential face (female tapered face) 12 of the tubular portion 11 may or may not be fitted to the outer circumferential face (male tapered face) 922 of the male member 921 liquid-tightly when the liquid-tight seal 29 is formed between the cylindrical portion 17 and the male member 921 (see FIG. 5). When the inner circumferential face 12 is fitted to the outer circumferential face 922 liquid-tightly, a liquid-tight seal is also formed between the inner circumferential face 12 and the outer circumferential face 922. Since a plurality of seals are formed between the female connector 2 and the male connector 100, this configuration is advantageous to prevent the liquid from passing through a slight gap between the female connector 2 and the male connector 100 to leak outside when the liquid pressure is increased. When the annular protrusion 21 is made from a soft material, it is possible to simultaneously achieve the seal 29 at the annular protrusion 21 and the seal between the inner circumferential face 12 and the outer circumferential face 922 with relatively relaxed accuracy.

The annular protrusion 21 of Embodiment 2 may be applied to the female connector 1 of Embodiment 1. In this case, a plurality of the liquid-tight seals 19 and 29 are formed between the cylindrical portion 17 and the male member 921. This configuration is advantageous to reduce a possibility of the liquid leakage to the space la even if the liquid pressure is increased.

Embodiment 2 is the same as Embodiment 1 except for the above points. The description of Embodiment 1 can be appropriately applied to Embodiment 2.

### Embodiment 3

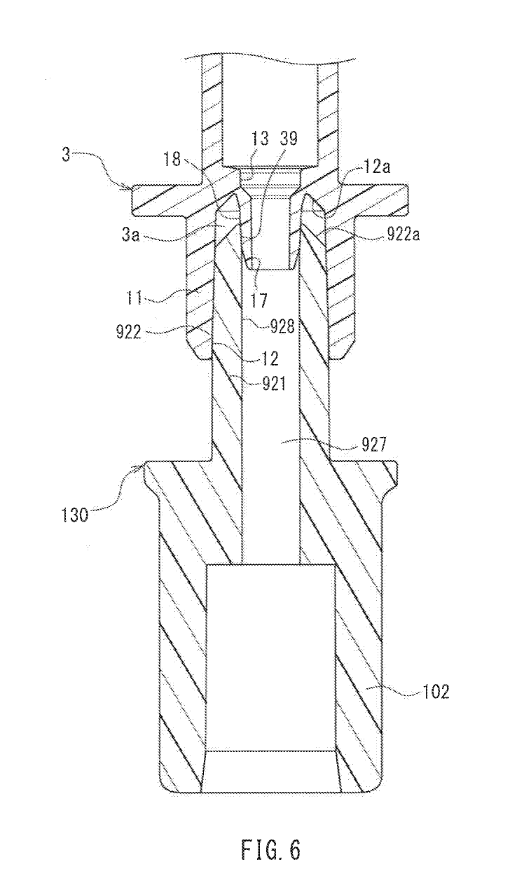

FIG. 6 is a cross-sectional view showing a state in which a female connector 3 according to Embodiment 3 of the present invention is connected to a male connector 130. In the drawings referred to hereinafter, the same reference numerals as those in the drawings of Embodiment 1 are assigned to members corresponding to the members illustrated in the drawings referred to in Embodiment 1. Although the explanations of such members are not repeated in this embodiment, they should be taken into account appropriately. Hereinafter, Embodiment 3 will be described mainly in terms of differences from Embodiment 1.

The female connector 3 of Embodiment 3 is different from the female connector 1 of Embodiment 1 in that the spiral protrusion 15 (see FIGS. 1A and 1B) is not provided on the outer circumference face of the tubular portion 11. The male connector 130 is different from the male connector 100 described in Embodiments 1, 2 (see FIGS. 2A and 2B) and the male connector 920 compliant with ISO 80369-3 (see FIGS. 13A and 13B) in that the male connector 130 includes neither the outer tube 923 nor the female threading 925. In other words, Embodiment 3 does not include screw lock mechanisms for maintaining the connected state of the female connector 3 and the male connector 130.

As shown in FIG. 6, the cylindrical portion 17 of the female connector 3 is inserted in the male member 921 of the male connector 130. A male tapered face is provided on the outer circumferential face 18 of the cylindrical portion 17, and the inner circumferential face 928 of the male member 921 is a circular cylindrical face. Therefore, similarly to Embodiment 1, the male tapered face of the cylindrical portion 17 and the tip portion of the inner circumferential face 928 of the male member 921 (i.e., the opening edge of the channel 927 on the tip side) are fitted to each other, and thereby a liquid-tight seal 39 is formed therebetween. Consequently, the cylindrical portion 17 and the male member 921 communicate with each other liquid-tightly.

The male member 921 of the male connector 130 is inserted in the tubular portion 11 of the female connector 3.

When the female connector 3 and the male connector 130 are in the connected state as shown in FIG. 6, a liquid (e.g., an enteral nutrient) flows from the female connector 3 to the male connector 130 (downward in FIG. 6). Similarly to Embodiment 1, since the liquid-tight seal 39 is formed between the cylindrical portion 17 and the male member 921, the liquid will not flow into a space 3a surrounded by the tubular portion 11 and the cylindrical portion 17 and the male member 921. Accordingly, when the female connector 3 and the male connector 130 are separated later, the liquid will not be attached to the base-end inner circumferential face 12a of the tubular portion 11 or the tip outer circumferential face 922a of the male member 921. Thus, it is possible to reduce the amount of the liquid to be attached to the female connector 3 and the male connector 130 after separation of the female connector 3 and the male connector 130, as compared with the case of using the female connector 910 shown in FIGS. 12A and 12B. Accordingly, similarly to Embodiment 1,an accurate amount of the liquid as measured can be administered to a patient.

In Embodiment 3, the inner circumferential face 12 of the tubular portion 11 may or may not be fitted to the outer circumferential face 922 of the male member 921 liquid-tightly when the liquid-tight seal 39 is formed between the cylindrical portion 17 and the male member 921. The liquid-tight fitting of the inner circumferential face 12 and the outer circumferential face 922 is advantageous to maintain the connected state of the female connector 3 and the male connector 130 stably.

Embodiment 3 is the same as Embodiment 1 except for the above points. The description of Embodiment 1 can be appropriately applied to Embodiment 3.

Although not illustrated, the annular protrusion 21 described in Embodiment 2 may be provided on the cylindrical portion 17 of the female connector 3 of Embodiment 3. In this case, the annular protrusion 21 comes into close contact with the inner circumferential face 928 of the male member 921, thereby forming the liquid-tight seal 29 (see FIG. 5) between the annular protrusion 21 and the inner circumferential face 928. The description of Embodiment 2 is appropriately applied to the female connector 3 provided with the annular protrusion 21.

### Embodiment 4

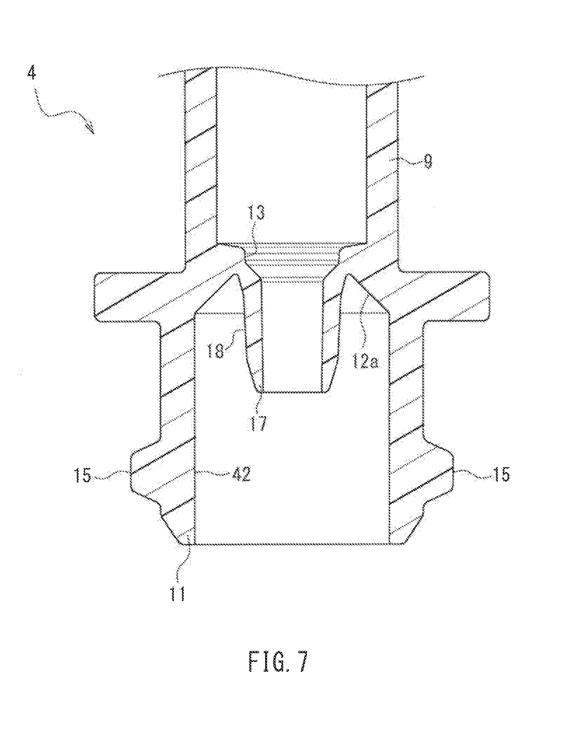

FIG. 7 is a cross-sectional view of a female connector 4 according to Embodiment 4 of the present invention. In the drawings referred to hereinafter, the same reference numerals as those in the drawings of Embodiment 1 are assigned to members corresponding to the members illustrated in the drawings referred to in Embodiment 1. Although the explanations of such members are not repeated in this embodiment, they should be taken into account appropriately. Hereinafter, Embodiment 4 will be described mainly in terms of differences from Embodiment 1.

In the female connector 4 of Embodiment 4, an inner circumference face 42 of the tubular portion 11 is a circular cylindrical face whose inner diameter is constant along the lengthwise direction of the tubular portion 11 (i.e., the longitudinal direction of FIG. 7). The inner diameter of the inner circumferential face 42 is larger than the maximum outer diameter of the outer circumferential face 922 of the male member 921 of the male connector 100 (see FIGS. 2A and 2B) to be connected to the female connector 4.

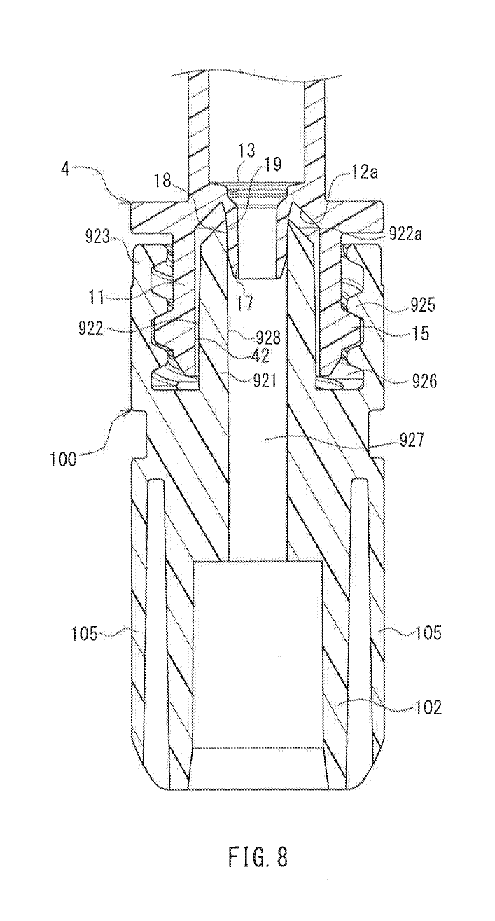

Similarly to Embodiment 1, the female connector 4 can be connected to the male connector 100 (see FIGS. 2A and 2B). FIG. 8 is a cross-sectional view showing a state in which the female connector 4 is connected to the male connector 100. Similarly to Embodiment 1, the cylindrical portion 17 of the female connector 4 is inserted in the male member 921 of the male connector 100. The male tapered face provided on the outer circumferential face 18 of the cylindrical portion 17 and the tip portion of the inner circumferential face 928 of the male member 921 (i.e., the opening edge of the channel 927 on the tip side) are fitted to each other, and thereby the liquid-tight seal 19 is formed therebetween. Consequently, the cylindrical portion 17 and the male member 921 communicate with each other liquid-tightly. The spiral protrusion 15 of the female connector 4 is screwed with the female threading 925 of the male connector 100.

Unlike Embodiment 1, since the inner circumferential face 42 of the tubular portion 11 is larger than the outer diameter of the outer circumferential face 922 of the male member 921, the inner circumferential face 42 and the outer circumferential face 922 are spaced apart from each other in the radial direction.

If the inner circumference face of the tubular portion 11 is the inner circumferential face 912 of the female connector 910 shown in FIGS. 12A and 12B unlike this Embodiment 4, there is a possibility that the inner circumferential face 912 and the outer circumferential face 922 are fitted to each other before the fitting of the outer circumferential face 18 of the cylindrical portion 17 and the inner circumferential face 928 of the male member 921. In this case, the male member 921 cannot be inserted deeper into the tubular portion 11, and the liquid-tight seal 19 cannot be formed between the outer circumferential face 18 of the cylindrical portion 17 and the inner circumferential face 928 of the male member 921. As described as the first configuration example in Embodiment 1, although it is possible to simultaneously achieve the fitting of the outer circumferential face 18 of the cylindrical portion 17 and the inner circumferential face 928 of the male member 921 and the fitting of the inner circumferential face 912 and the outer circumferential face 922, each of fitting portions needs to have high accuracy to achieve this.

In Embodiment 4, the inner circumferential face 42 of the tubular portion 11 is not fitted to the outer circumferential face 922 of the male member 921. The inner circumferential face 42 does not adversely affect the formation of the seal 19 or its liquid tightness. The inner circumferential face 42 is advantageous to improve the liquid tightness of the seal 19 formed at a fitting portion of the outer circumferential face 18 of the cylindrical portion 17 and the inner circumferential face 928 of the male member 921 because the inner circumferential face 42 can intensively exert on this fitting portion a bonding force of the male connector 100 with respect to the female connector 4 that is generated by screwing the female threading 925 with the spiral protrusion 15 (a force that attracts the male connector 100 toward the female connector 4). This is also advantageous to relax the accuracy of the inner circumferential face 42 of the tubular portion 11 as compared with the inner circumferential face 912.

In the above example, although the inner circumferential face 42 of the tubular portion 11 is a circular cylindrical face whose inner diameter is constant, the present invention is not limited to this. For example, the inner circumferential face 42 may be a tapered face whose inner diameter increases toward the tip (a so-called female tapered face). In this case, the inner diameter of the female tapered face of the inner circumferential face 42 needs to be larger than the outer diameter of the outer circumferential face 922 of the male member 921 so that the inner circumferential face 42 is not fitted to the outer circumferential face 922 of the male member 921 when the liquid-tight seal 19 is formed between the outer circumferential face 18 of the cylindrical portion 17 and the inner circumferential face 928 of the male member 921.

In Embodiment 4, as described above, the inner circumferential face 42 of the tubular portion 11 is configured so that the inner circumferential face 42 of the tubular portion 11 and the outer circumferential face 922 of the male member 921 are spaced apart from each other (i.e., so that they are not fitted to each other) when the liquid-tight seal 19 is formed between the cylindrical portion 17 and the male member 921. Thus, it is possible to improve the liquid tightness of the seal 19 between the cylindrical portion 17 and the male member 921.

The liquid flows through the cylindrical portion 17 and the male member 921 that are communicating with each other liquid-tightly. The seal 19 formed prevents the liquid from being attached to the base-end inner circumferential face 12a of the tubular portion 11 or the tip outer circumferential face 922a of the male member 921 or flowing into the gap 926 by passing through between the cylindrical portion 17 and the male member 921. Therefore, Embodiment 4 achieves effects similar to those of Embodiment 1.

The inner circumferential face 42 of Embodiment 4 may be applied to the female connectors 2 and 3 of Embodiments 2 and 3, respectively.

Embodiment 4 is the same as Embodiment 1 except for the above points. The description of Embodiment 1 can be appropriately applied to Embodiment 4.

### Embodiment 5

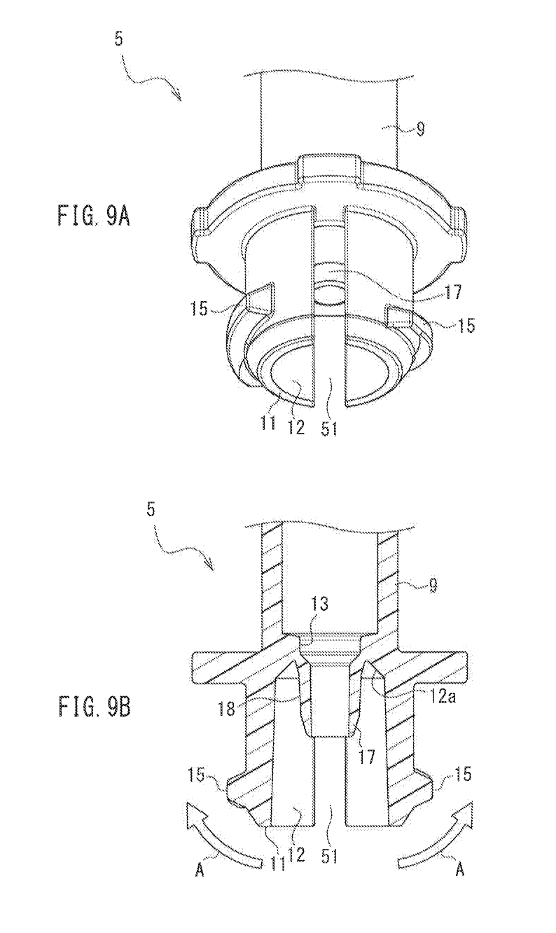

FIG. 9A is a perspective view of a female connector 5 according to Embodiment 5 of the present invention, and FIG. 9B is a cross-sectional view thereof. In the drawings referred to hereinafter, the same reference numerals as those in the drawings of Embodiment 1 are assigned to members corresponding to the members illustrated in the drawings referred to in Embodiment 1. Although the explanations of such members are not repeated in this embodiment, they should be taken into account appropriately. Hereinafter, Embodiment 5 will be described mainly in terms of differences from Embodiment 1.

In the female connector 5 of Embodiment 5, two slits 51 are formed in the tubular portion 11. The slits 51 are parallel to the lengthwise direction of the tubular portion 11 (i.e., the center axis direction of the female connector 5) and extend from the tip of the tubular portion 11 to the base end of the tubular portion 11. The two slits 51 are arranged symmetrically with respect to the cylindrical portion 17 in an area of the tubular portion 11 where the spiral protrusion 15 is not provided. Since the slits 51 are formed in the tubular portion 11, it is possible to elastically bend and deform the tubular portion 11 in directions indicated by arrow A in FIG. 9B, and to elastically deform the tubular portion 11 so that the curvature of the inner circumferential face 12 increases in the circumferential direction. When the tubular portion 11 is deformed like this, the diameter of the tubular portion 11 is enlarged, and the inner diameter of the inner circumferential face 12 is increased.

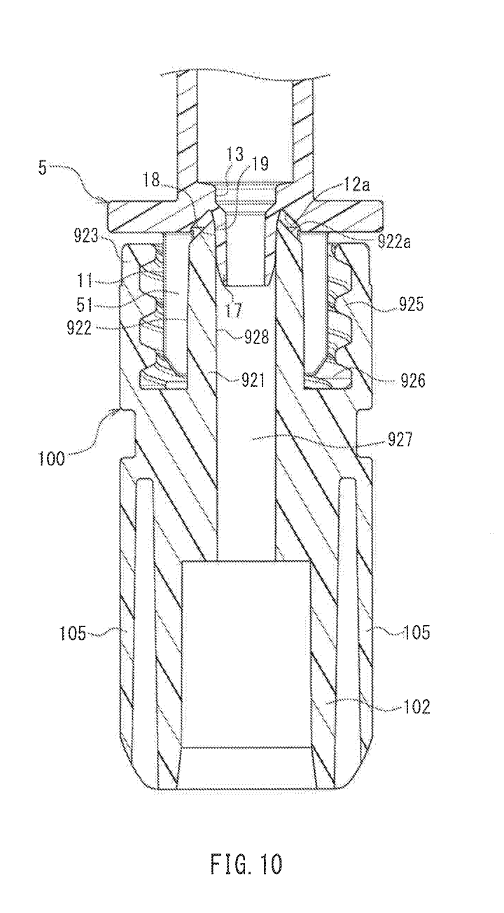

Similarly to Embodiment 1, the female connector 5 can be connected to the male connector 100 (see FIGS. 2A and 2B). FIG. 10 is a cross-sectional view showing a state in which the female connector 5 is connected to the male connector 100. The cross section of FIG. 10 passes through the slits 51 and is orthogonal to the cross section of FIG. 9B. Similarly to Embodiment 1, the cylindrical portion 17 of the female connector 5 is inserted in the male member 921 of the male connector 100. The male tapered face of the outer circumferential face 18 of the cylindrical portion 17 and the tip portion of the inner circumferential face 928 of the male member 921 (i.e., the opening edge of the channel 927 on the tip side) are fitted to each other, and thereby the liquid-tight seal 19 is formed therebetween. Consequently, the cylindrical portion 17 and the male member 921 communicate with each other liquid-tightly. Although not illustrated in FIG. 10, the spiral protrusion 15 of the female connector 4 is screwed with the female threading 925 of the male connector 100.

In a process of connecting the female connector 5 to the male connector 100, the inner circumferential face 12 of the tubular portion 11 may collide with the outer circumferential face 922 of the male member 921 before the fitting of the outer circumferential face 18 of the cylindrical portion 17 and the inner circumferential face 928 of the male member 921. Even in this case, since the tubular portion 11 is elastically deformable to enlarge, the male member 921 can be inserted deeper into the tubular portion 11. Thus, it is possible to fit the inner circumferential face 928 of the male member 921 to the outer circumferential face 18 of the cylindrical portion 17 and form the liquid-tight seal 19 therebetween. It is also possible to relax the accuracy of the inner circumferential face 12 of the tubular portion 11.

In Embodiment 5, the inner circumference face of the tubular portion 11 may be the inner circumferential face 912 of the female connector 910 shown in FIGS. 12A and 12B. Even in this case, the liquid-tight seal 19 can be formed between the cylindrical portion 17 and the male member 921 by deformation of the tubular portion 11 as described above.

In Embodiment 5, the number, the arrangement, and the size (width, depth) of the slit 51 are not limited to the above example, and they are determined appropriately. The number of the slit 51 is preferably two or more, more preferably three or more from the viewpoint of easy deformation of the tubular portion 11. In this case, it is preferred that a plurality of the slits 51 be arranged at equal intervals in the circumferential direction. As to the depth of the slit 51, as shown in FIG. 10, it is preferred that the slit 51 extend further to the small diameter portion 13 side than an area where the male member 921 may come into contact when the liquid-tight seal 19 is formed.

The liquid flows through the cylindrical portion 17 and the male member 921 that are communicating with each other liquid-tightly. The seal 19 formed prevents the liquid from being attached to the base-end inner circumferential face 12a of the tubular portion 11 or the tip outer circumferential face 922a of the male member 921 or flowing into the gap 926 by passing through between the cylindrical portion 17 and the male member 921. Therefore, Embodiment 5 achieves effects similar to those of Embodiment 1.

The slit 51 of Embodiment 5 may be applied to the female connectors 2 to 4 of Embodiments 2 to 4, respectively. When the slit 51 is applied to the female connector 3 of Embodiment 3 (see FIG. 6), the liquid-tight seal 39 can be formed reliably between the cylindrical portion 17 and the male member 921 while the inner circumferential face 12 of the tubular portion 11 and the outer circumferential face 922 of the male member 921 are fitted to each other.

Embodiment 5 is the same as Embodiment 1 except for the above points. The description of Embodiment 1 can be appropriately applied to Embodiment 5.

### Embodiment 6

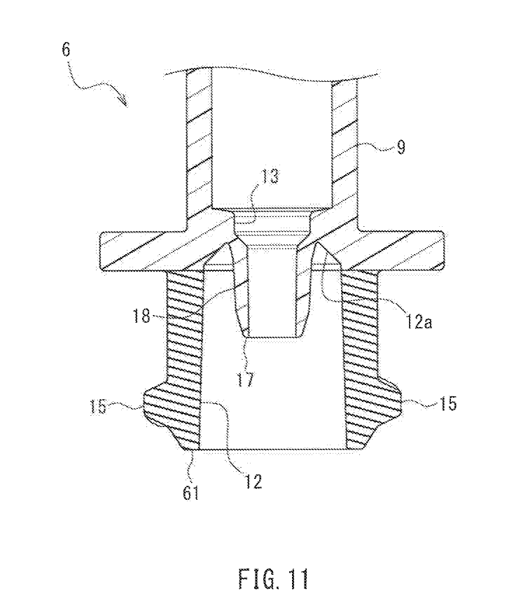

FIG. 11 is a cross-sectional view of a female connector 6 according to Embodiment 6 of the present invention. In the drawings referred to hereinafter, the same reference numerals as those in the drawings of Embodiment 1 are assigned to members corresponding to the members illustrated in the drawings referred to in Embodiment 1. Although the explanations of such members are not repeated in this embodiment, they should be taken into account appropriately. Hereinafter, Embodiment 6 will be described mainly in terms of differences from Embodiment 1.

In the female connector 6 of Embodiment 6, a tubular portion 61 including the spiral protrusion 15 is constituted by a relatively soft material as compared with the material constituting the portions other than the tubular portion 61. Examples of the soft material constituting the tubular portion 61 include, but are not limited to, the same soft materials as those described in Embodiment 1 as the soft materials that can constitute the cylindrical portion 17.

The tubular portion 61 made from a soft material is formed integrally with the other portions of the female connector 6 as a single piece. The method of integration is not limited, and may be, e.g., the coinjection molding method or the connection method described in Embodiment 1.

Similarly to Embodiment 1 (see FIGS. 3A and 3B), the female connector 6 can be connected to the male connector 100 (see FIGS. 2A and 2B).

In a process of connecting the female connector 6 to the male connector 100, the inner circumferential face 12 of the tubular portion 61 may collide with the outer circumferential face 922 of the male member 921 before the fitting of the outer circumferential face 18 of the cylindrical portion 17 and the inner circumferential face 928 of the male member 921. Even in this case, since the tubular portion 61 made from a soft material appropriately deform elastically by a pressing force applied from the male member 921, the male member 921 can be inserted deeper into the tubular portion 61. Therefore, similarly to Embodiment 1, it is possible to fit the inner circumferential face 928 of the male member 921 to the outer circumferential face 18 of the cylindrical portion 17 and form the liquid-tight seal 19 therebetween.

In Embodiment 6, the inner circumference face of the tubular portion 61 may be the inner circumferential face 912 of the female connector 910 shown in FIGS. 12A and 12B. Even in this case, since the tubular portion 61 deforms appropriately as described above, the liquid-tight seal 19 can be formed between the cylindrical portion 17 and the male member 921.

The liquid-tight seal 19 prevents the liquid from being attached to the base-end inner circumferential face 12a of the tubular portion 11 or the tip outer circumferential face 922a of the male member 921 or flowing into the gap 926 by passing through between the cylindrical portion 17 and the male member 921. Therefore, Embodiment 6 achieves effects similar to those of Embodiment 1.

The tubular portion 61 can form a liquid-tight seal between the tubular portion 61 and the outer circumferential face 922 by coming into close contact with the outer circumferential face 922 of the male member 921 while appropriately changing its shape in accordance with the male member 921. This seal is advantageous to prevent the liquid that has leaked out from the seal 19 between the cylindrical portion 17 and the male member 921 from leaking outside when the liquid pressure is increased.

Since the tubular portion 61 can be deformed relatively easily, it is possible to relax the accuracy of the tubular portion 61.

The tubular portion 61 made from a soft material of Embodiment 6 may be applied to the female connectors 2 to 5 of Embodiments 2 to 5, respectively. Constituting the tubular portion 11 of the female connector 3 of Embodiment 3 by a soft material allows the tubular portion 11 to be fitted to the male member 921, and thus is advantageous to maintain the connected state of the female connector 3 and the male connector 130 stably.

Embodiment 6 is the same as Embodiment 1 except for the above points. The description of Embodiment 1 can be appropriately applied to Embodiment 6.

In Embodiments 1 to 6 described above, the liquid-tight seals 19, 29 and 39 are formed by inserting the cylindrical portion 17 into the male member 921 (see FIGS. 3B, 5, 6, 8, and 10). However, the present invention is not limited to this. For example, a liquid-tight seal may be formed between the tip of the cylindrical portion 17 and the tip of the male member 921 by butting together the cylindrical portion 17 and the male member 921 in the lengthwise direction. This case also achieves the above effects of the present invention. Further, the inner diameter of the cylindrical portion 17 can be set larger than those in Embodiments 1 to 6. This is advantageous to prevent a decrease of the flow resistance of the liquid in the cylindrical portion 17. In order to improve the liquid tightness of the seal formed between the tip of the cylindrical portion 17 and the tip of the male member 921, it is advantageous that at least the tip of the cylindrical portion 17 is constituted by any of the above soft materials. In the configuration in which the cylindrical portion 17 and the male member 921 are butted together to form a seal therebetween, in order to apply a compressive force between the cylindrical portion 17 and the male member 921, it is preferred that (1) the female connector and the male connector include screw lock mechanisms (the spiral protrusion 15 and the female threading 925), or that (2) the tubular portion of the female connector be fitted to the male member of the male connector.

The female connectors of the present invention are not necessarily compliant with ISO 80369-3. Further, the male connectors to which the female connectors of the present invention are connected are not necessarily compliant with ISO 80369-3.

### INDUSTRIAL APPLICABILITY

The present invention is applicable as a female connector that includes a tubular portion to which a male member of a male connector is insertable, and there are no limitations on its application. Particularly, the present invention is preferably applicable as a medical female connector, more preferably as a female connector used for enteral feeding, and particularly preferably as a female connector to be connected to a male connector compliant with ISO 80369-3. The female connector of the present invention is suitable as a female connector to be connected to a male connector attached to the upstream end of a catheter that is inserted in a patient's body for enteral feeding.

### DESCRIPTION OF REFERENCE NUMERALS

1, 2, 3, 4, 5, 6 Female connector

11, 61 Tubular portion

12, 42 Inner circumferential face of the tubular portion

15 Spiral protrusion

17 Cylindrical portion

18 Outer circumferential face of the cylindrical portion

19 Liquid-tight seal

21 Annular protrusion (O-ring)

28 Outer circumferential face of the cylindrical portion

29 Liquid-tight seal

39 Liquid-tight seal

51 Slit

100, 130 Male connector

921 Male member

922 Outer circumferential face of the male member

923 Outer tube

925 Female threading

928 Inner circumferential face of the male member

1. A female connector that is connectable to a male connector comprising a cylindrical male member, the female connector comprising:
a tubular portion having a hollow circular cylindrical shape to which the male member is insertable; and
a cylindrical portion having a hollow circular cylindrical shape that is coaxial with the tubular portion and surrounded by the tubular portion,
wherein the cylindrical portion is configured so that, when the male member is inserted into the tubular portion, a liquid-tight seal is formed between the cylindrical portion and the male member, and the cylindrical portion and the male member communicate with each other.

2. The female connector according to claim 1, wherein the liquid-tight seal is formed between an outer circumference face of the cylindrical portion and an inner circumference face of the male member.

3. The female connector according to claim 1,
wherein an outer circumference face of the cylindrical portion includes a male tapered face whose outer diameter decreases toward a tip, and
the liquid-tight seal is formed between the male tapered face and an inner circumference face of the male member.

4. The female connector according to claim 1, wherein the cylindrical portion is made from a soft material.

5. The female connector according to claim 1,
wherein an outer circumference face of the cylindrical portion has an annular protrusion that is continuous in a circumferential direction of the cylindrical portion, and
the liquid-tight seal is formed between the annular protrusion and an inner circumference face of the male member.

6. The female connector according to claim 5, wherein the annular protrusion is an O-ring that is mounted on the outer circumference face of the cylindrical portion.

7. The female connector according to claim 1, wherein when the liquid-tight seal is formed, an inner circumference face of the tubular portion is not fitted to an outer circumference face of the male member liquid-tightly.

8. The female connector according to claim 1, wherein when the liquid-tight seal is formed, an inner circumference face of the tubular portion and an outer circumference face of the male member are spaced apart from each other.

9. The female connector according to claim 1, wherein the tubular portion includes a slit so as to be elastically deformable to enlarge its diameter.

10. The female connector according to claim 1, wherein the tubular portion is made from a soft material.

11. The female connector according to claim 1, wherein when the liquid-tight seal is formed, an inner circumference face of the tubular portion is fitted to an outer circumference face of the male member liquid-tightly.

12. The female connector according to claim 1,
wherein the male connector further comprises an outer tube that surrounds the male member, and female threading that is provided on the outer tube so as to face the male member, and
the female connector further comprises a spiral protrusion that is provided on an outer circumference face of the tubular portion so as to be screwed with the female threading.
